(19) United States
(12) Patent Application Publication
(10) Pub. No.: US 2015/0182967 A1
(43) Pub. Date: Jul. 2, 2015

(54) PRINTED CIRCUIT BOARD DESIGNS FOR LAMINATED MICROFLUIDIC DEVICES

(71) Applicant: Canon U.S. Life Sciences, Inc., Rockville, MD (US)

(72) Inventors: Johnathan S. COURSEY, Rockville, MD (US); Hongye LIANG, Clarksville, MD (US)

(73) Assignee: CANON U.S. LIFE SCIENCES, INC., Rockville, MD (US)

(21) Appl. No.: 14/586,619

(22) Filed: Dec. 30, 2014

Related U.S. Application Data
(60) Provisional application No. 61/922,795, filed on Dec. 31, 2013.

(51) Int. Cl. B01L 7/00; C12Q 1/68

(52) U.S. Cl. CPC B01L 7/52; C12Q 1/6806

(57) ABSTRACT

A microfluidic device is disclosed including a printed circuit board (PCB) and a microfluidic layer attached to the PCB. The microfluidic layer may include a microfluidic feature. The PCB may include laminated non-conductive and conductive layers. The PCB may also include an electronic component embedded in the laminated non-conductive and conductive layers. A non-conductive layer of the non-conductive layers may be configured to fluidically isolate the electronic component from fluid in the microfluidic feature. The electronic component may be connected to a conductor of a conductive layer of the conductive layers. The PCB may have a fiberglass core or a metal core, which may spread heat to the microfluidic feature. One or more of the conductive layers may be made with heavy copper or extreme copper, and the heavy or extreme copper may spread heat to the microfluidic feature.

### CROSS REFERENCE TO RELATED APPLICATION

This application claims the benefit of priority to U.S. Provisional Application Ser. No. 61/922,795, filed on Dec. 31, 2013, which is incorporated herein by reference in its entirety.

### BACKGROUND

1. Field of Invention

The present invention relates to microfluidic devices. More specifically, embodiments of the present invention relate to microfluidic devices including a microfluidic layer attached to a printed circuit board.

2. Discussion of the Background

The detection of nucleic acids is central to medicine, forensic science, industrial processing, crop and animal breeding, and many other fields. The ability to detect disease conditions (e.g., cancer), infectious organisms (e.g., HIV), genetic lineage, genetic markers, and the like, is ubiquitous technology for disease diagnosis and prognosis, marker assisted selection, identification of crime scene features, the ability to propagate industrial organisms and many other techniques. Determination of the integrity of a nucleic acid of interest can be relevant to the pathology of an infection or cancer.

One of the most powerful and basic technologies to detect small quantities of nucleic acids is to replicate some or all of a nucleic acid sequence many times, and then analyze the amplification products. Polymerase chain reaction (PCR) is a well-known technique for amplifying deoxyribonucleic acid (DNA). With PCR, one can produce millions of copies of DNA starting from a single template DNA molecule. PCR includes phases of “denaturation,” “annealing,” and “extension.” These phases are part of a cycle which is repeated a number of times so that at the end of the process there are enough copies to be detected and analyzed. For general details concerning PCR, see Sambrook and Russell, Molecular Cloning—A Laboratory Manual (3rd Ed.), Vols. 1-3, Cold Spring Harbor Laboratory, Cold Spring Harbor, N.Y. (2000); Current Protocols in Molecular Biology, F. M. Ausubel et al., eds., Current Protocols, a joint venture between Greene Publishing Associates, Inc. and John Wiley & Sons, Inc., (supplemented through 2005) and PCR Protocols A Guide to Methods and Applications, M. A. Innis et al., eds., Academic Press Inc. San Diego, Calif. (1990).

The PCR process phases of denaturing, annealing, and extension occur at different temperatures and cause target DNA molecule samples to replicate themselves. Temperature cycling (thermocyling) requirements vary with particular nucleic acid samples and assays. In the denaturing phase, a double stranded DNA (dsDNA) is thermally separated into single stranded DNA (ssDNA). During the annealing phase, primers are attached to the single stranded DNA molecules. Single stranded DNA molecules grow to double stranded DNA again in the extension phase through specific bindings between nucleotides in the PCR solution and the single stranded DNA. Typical temperatures are 95° C. for denaturing, 55° C. for annealing, and 72° C. for extension. The temperature is held at each phase for a certain amount of time which may be a fraction of a second up to a few tens of seconds. The DNA is doubled at each cycle, and it generally takes 20 to 40 cycles to produce enough DNA for certain applications. To have good yield of target product, one has to accurately control the sample temperatures at the different phases to a specified degree.

More recently, a number of high throughput approaches to performing PCR and other amplification reactions have been developed, e.g., involving amplification reactions in microfluidic devices, as well as methods for detecting and analyzing amplified nucleic acids in or on the devices. Thermal cycling of the sample for amplification is usually accomplished in one of two methods. In the first method, the sample solution is loaded into the device and the temperature is cycled in time, much like a conventional PCR instrument. In the second method, the sample solution is pumped continuously through spatially varying temperature zones. See, for example, Lagally et al. (Analytical Chemistry 73:565-570 (2001)), Kopp et al. (Science 280:1046-1048 (1998)), Park et al. (Analytical Chemistry 75:6029-6033 (2003)), Hahn et al. (WO 2005/075683), Enzelberger et al. (U.S. Pat. No. 6,960,437) and Knapp et al. (U.S. Patent Application Publication No. 2005/0042639). Many detection methods require a determined large number of copies (millions, for example) of the original DNA molecule, in order for the DNA to be characterized (e.g., via a melting curve analysis).

Microfluidic devices for performing these chemical, biological, or other reactions (e.g., microfluidic devices for performing PCR amplification and/or high resolution melt analysis) are known. See, e.g., U.S. Pat. Nos. 7,629,124 and 7,906,319. Often these microfluidic devices feature one or more thermal control elements that are used to subject reactants to a desired thermal profile. Some microfluidic devices have incorporated elements of the microfluidic device in printed circuit boards (PCBs). See, e.g., Dr. Leanna M. Levine, Rapid prototyping of microfluidic devices with PLT, MICROmanufacturing, Volume 3, Issue 6 (November/December 2010); http://www.micromanufacturing.com/content/rapid-prototyping-devices-plt; Ortiz et al., A Cancer Diagnostics Biosensor System Based on Micro- and Nano-technologies, Nano-Net, Volume 20, pp. 169-177 (2009); Press Release, Panasonic, Development of fully automatic compact constitution diagnostic genetic testing chip (Feb. 14, 2013) (available at http://panasonic.co.jp/corp/news/official.data/data.dir/2013/02/jn130214-1/jn130214-1.html); R. B. Oueslati et al., PCB-Integrated Heat Exchanger for Cooling Electronics using Microchannels Fabricated with the Direct-Write Method, IEEE Transactions on Components and Packaging Technologies, Vol. 31, Issue 4, pp. 869-874 (Dec. 2008); E. J. Vardaman et al., Market Drivers for Embedded Components Packaging, TechSearch International (2013) (available at http://www.semi.org/eu/sites/semi.org/files/docsNardamanEmbMktHD.pdf); http://www.saturnelectronics.com/products capabilities/; http://www.4pcb.com/Capabilities-Brochure-NOV2013-FINAL.pdf; William J. Borland & Saul Ferguson, Embedded Passive Components in Printed Wiring Boards, a Technology Review, CircuiTree Magazine (March 2001); Markus Leitgeb & Christopher Ryder, SMT Manufacturing and Reliability in PCB Cavities, PCB 007 (Jan. 8, 2013) (available at http://www.pcb007.com/pages/zone.cgi?artcatid=0&a=88968&artid=88968&pg=3); http://www.saturnelectronics.com/products_capabilities/cavity_board.html. While microfluidic devices have incorporated elements of the microfluidic device in PCBs, these prior efforts lack, among several deficiencies, an efficient combination of techniques so that benefits of advancements in electronics can be combined with the emerging applications of microfluidics.

There is thus a need in the art for an improved microfluidic device capable of performing one or more reactions to amplify and/or characterize nucleic acids and methods of manufacturing these microfluidic devices.

### SUMMARY

The present invention relates to microfluidic devices including a microfluidic layer attached to a printed circuit board. In one aspect of the invention, a microfluidic device comprises a microfluidic layer including a microfluidic feature, and a PCB to which the microfluidic layer is attached. In one embodiment, the PCB comprises electrically non-conductive layers, electrically conductive layers laminated with the non-conductive layers, and an electronic component embedded in the laminated non-conductive and conductive layers, wherein a non-conductive layer of the non-conductive layers is configured to fluidically isolate the electronic component from fluid in the microfluidic feature, and the electronic component is connected to a conductor of a conductive layer of the conductive layers.

In one embodiment, the PCB further comprises a recess in one or more layers of the laminated non-conductive and conductive layers, and the electronic component is embedded in the recess. In some embodiments, the non-conductive layer configured to fluidically isolate the electronic component from fluid in the microfluidic feature is a conformal coating. In other embodiments, the microfluidic layer is attached to the conformal coating and the conformal coating is configured to planarize a surface of the PCB to which the microfluidic layer is attached.

In one embodiment, the electronic component may be a formed passive component, a placed discrete passive component, or a placed active component. In some embodiments, the electronic component may be, for example, a resistor, capacitor, diode, transistor, or integrated circuit. In some embodiments, the electronic component is configured to heat fluid in the microfluidic feature and may be large relative to the microfluidic feature.

In some embodiments, the electronic component may be a light source configured to emit light and irradiate the microfluidic feature. In some embodiments, the light source is configured to excite a fluorophore in the microfluidic feature. In other embodiments, the electronic component may be a photodetector configured to detect light received from the microfluidic feature. In some embodiments, the electronic component may be configured to measure the temperature of fluid in the microfluidic feature. In some embodiments, the microfluidic feature may include a microfluidic channel and/or a microwell.

In some embodiments, the electronic component may be located below the microfluidic feature. In some embodiments, the microfluidic device comprises a plurality of microfluidic layers, and any of the microfluidic layers may include a plurality of microfluidic features. In some embodiments, the PCB includes a plurality of electronic devices, which may include, for example, a light source and a photodetector. The light source and photodetector may be embedded in a recess in one or more layers of the laminated non-conductive and conductive layers. In some embodiments, the recess may include includes one or more optical filters.

In some embodiments, one or more of the conductive layers may comprise copper and have greater than or equal to a 3 oz thickness. In some embodiments, the microfluidic layer may be attached to the PCB using, for example, a solvent, an adhesive or thermal bonding. In some embodiments, the PCB may be a metal core PCB.

In another aspect of the invention, the microfluidic device comprises a microfluidic layer including one or more microfluidic features and a metal core PCB to which the microfluidic layer is attached. In one embodiment, the PCB may comprise electrically non-conductive layers, electrically conductive layers laminated with the non-conductive layers, and a metal core configured to spread heat to the one or more microfluidic features. In some embodiments, the PCB may comprise a component connected to the metal core and configured to provide the heat spread by the metal core. In some embodiments, the component may be embedded in the laminated non-conductive and conductive layers of the PCB. In some embodiments, the heat spread by the metal core is provided by a component external to the microfluidic device.

In another aspect of the invention, a method of manufacturing a microfluidic device comprises embedding an electronic component in laminated electrically non-conductive layers and electrically conductive layers of a PCB, wherein the electronic component is connected to a conductor of a conductive layer of the conductive layers, and attaching a microfluidic layer including a microfluidic feature to the PCB, and wherein the electronic component is fluidically isolated from fluid in the microfluidic feature by a non-conductive layer of the non-conductive layers.

In one embodiment, embedding the electronic component may comprise forming a recess in one or more layers of the laminated non-conductive and conductive layers, and embedding the electronic component in the recess. In some embodiments, embedding the electronic component may comprise forming a conformal coating on the PCB, wherein the non-conductive layer configured to fluidically isolate the electronic component from fluid in the microfluidic feature is the conformal coating. In some embodiments, attaching the microfluidic layer to the PCB may comprise attaching the microfluidic layer to the conformal coating. In other embodiments, embedding the electronic component may comprise forming or placing the electronic component in the PCB.

Another aspect of the invention includes a method of heating fluid in a microfluidic feature of a microfluidic device comprising a microfluidic layer including the microfluidic feature and a PCB to which the microfluidic layer is attached. In one embodiment, the method may comprise using an electronic component embedded in laminated electrically non-conductive layers and electrically conductive layers of the PCB to heat fluid in the microfluidic feature of the microfluidic device, wherein the electronic component is fluidically isolated from the fluid in the microfluidic feature by a non-conductive layer of the non-conductive layers, and the electronic component is connected to a conductor of a conductive layer of the conductive layers. In some embodiments, the method may further comprise using the electronic component to measure the temperature of the fluid in the microfluidic feature.

Another aspect of the invention includes a method of irradiating fluid in a microfluidic feature of a microfluidic device comprising a microfluidic layer including the microfluidic feature and a PCB to which the microfluidic layer is attached. In one embodiment, the method may comprise using a light source embedded in laminated electrically non-conductive layers and electrically conductive layers of the PCB to emit light and irradiate the fluid in the microfluidic feature of the microfluidic device, wherein the light source is fluidically isolated from the fluid in the microfluidic feature by a non-conductive layer of the non-conductive layers, and the light source is connected to a conductor of a conductive layer of the conductive layers. In some embodiments, irradiating the fluid may comprise exciting a fluorophore in the microfluidic feature. In some embodiments, the method may further comprise using a photodetector embedded in the laminated non-conductive and conductive layers of the PCB to detect light received from the microfluidic feature.

Another aspect of the invention includes a method of manufacturing a microfluidic device. In one embodiment, the method may comprise attaching a microfluidic layer including a microfluidic feature to a metal core PCB comprising electrically non-conductive layers, electrically conductive layers laminated with the non-conductive layers, and a metal core configured to spread heat to the one or more microfluidic features.

Another aspect of the invention includes a method of spreading heat to fluid in one or more microfluidic features of a microfluidic device comprising a microfluidic layer including the one or more microfluidic feature and a PCB to which the microfluidic layer is attached. In one embodiment, the method may comprise using a metal core of the PCB to spread heat to the one or more microfluidic features, wherein the PCB includes the metal core, electrically non-conductive layers, and electrically conductive layers laminated with the non-conductive layers.

The above and other embodiments of the present invention are described below with reference to the accompanying drawings.

### BRIEF DESCRIPTION OF THE DRAWINGS

The accompanying drawings, which are incorporated herein and form part of the specification, illustrate various embodiments of the present invention. In the drawings, like reference numbers indicate identical or functionally similar elements. Additionally, the left-most digit(s) of the reference number identifies the drawing in which the reference number first appears.

FIG. 1 depicts a cross-sectional view of a microfluidic device including an electronic component embedded in a recess of a printed circuit board (PCB) embodying aspects of the present invention.

FIG. 2 depicts a cross-sectional view of a microfluidic device including an electronic component formed in a PCB embodying aspects of the present invention.

FIGS. 3A and 3B depict cross-sectional views of microfluidic devices including a fiberglass core PCB and a metal core PCB, respectively, embodying aspects of the present invention.

FIG. 4 depicts a cross-sectional view of a microfluidic device including an optical system embedded in a recess of a PCB embodying aspects of the present invention.

### DETAILED DESCRIPTION OF PREFERRED EMBODIMENTS

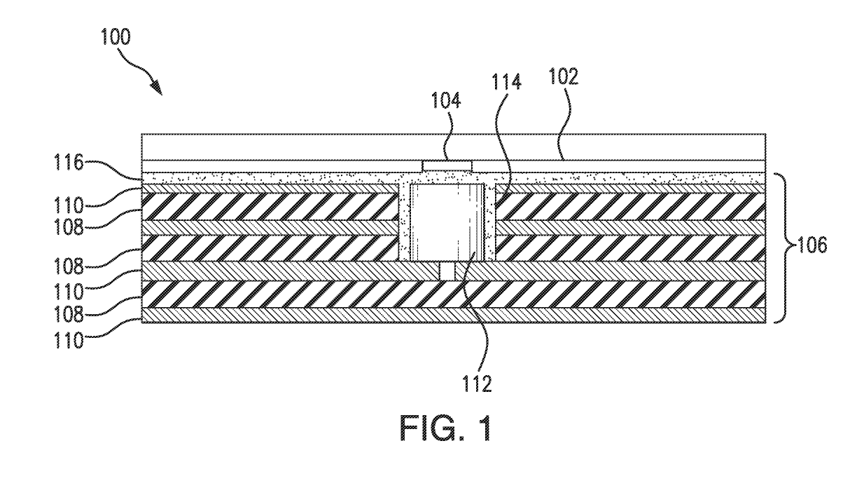

FIG. 1 is a cross-sectional view of a microfluidic device 100 embodying aspects of the present invention. In some non-limiting embodiments, the microfluidic device 100 may be a reaction chip configured to perform PCR thermal cycling and/or a thermal ramp for a melting curve analysis. In some embodiments, the microfluidic device 100 may include one or more microfluidic layers 102. In some embodiments, a microfluidic layer 102 may have one or more microfluidic features 104, such as, for example, one or more microfluidic channels and/or one or more micro-wells. In some embodiments, the microfluidic device 100 may include a printed circuit board (PCB) 106, and the microfluidic layer 102 may be attached (e.g., adhered, affixed, or laminated) to the PCB 106. In some non-limiting embodiments, the microfluidic layer 102 may be attached to the PCB using, for example and without limitation, solvent, thermal, or adhesive bonding.

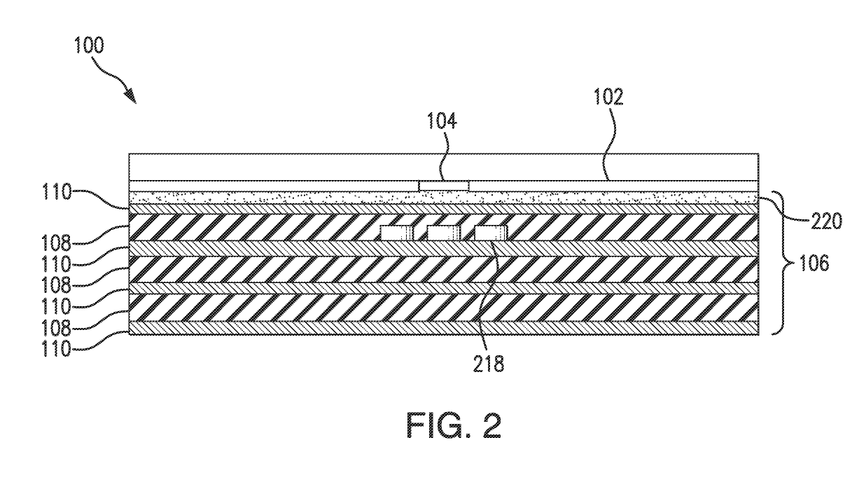

In some embodiments, the PCB 106 may include electrically non-conductive layers 108 and electrically conductive layers 110 laminated with the non-conductive layers 108. In some non-limiting embodiments, one or more of the non-conductive layers 108 may be a pre-preg layer (i.e., fiberglass impregnated with resin). However, this is not required, and, in some alternative embodiments, other materials may be used. In some embodiments, the microfluidic layer 102 may be attached to a non-conductive layer 108 of the PCB. In some non-limiting embodiments, the non-conductive layer 108 to which the microfluidic layer 102 is attached may be, for example and without limitation, a pre-preg layer, a conformal coating 116 (see FIG. 1), or an adhesion layer 220 (see FIG. 2). In some non-limiting embodiments, one or more non-conductive layers 108 (e.g., pre-preg, conformal, and/or an adhesion layers) may be added to the PCB 106 to create a flat/planar surface for attachment of the microfluidic layer 102.

In some non-limiting embodiments, one or more of the conductive layers 110 may be a copper layer. However, this is not required, and, in some alternative embodiments, other materials may be used. In some embodiments, one or more of the conductive layers 110 may include one or more conductors (i.e., signal traces or tracks). In some embodiments, the conductive layers 110 may function as signal, ground, or power planes. In some embodiments, the PCB 106 may include a standard stackup of non-conductive layers 108 and conductive layers 110, but this is not required, and, in alternative embodiments, the PCB 106 may include a non-standard stackup (e.g., a stackup including an odd number of conductive layers 110).

In some embodiments, the PCB 106 may include one or more electronic components 112 embedded in the laminated non-conductive and conductive layers 108 and 110. An electronic component 112 may be, for example and without limitation, a resistor, a capacitor, a temperature sensor (e.g., a resistance temperature detector (RTD)), a diode, a transistor, a light source (e.g., a light emitting diode (LED)), a photodetector (e.g., a photodiode, phototransistor, photoresistor or other photosensitive element), or an integrated circuit (IC). In some embodiments, a non-conductive layer 108 may be configured to fluidically isolate the electronic component 112 from fluid in the microfluidic feature 104. In some embodiments, the electronic component 112 may be connected to one or more conductors (i.e., signal traces or tracks) of a conductive layer 110.

In some embodiments, as shown in FIG. 1, the PCB 106 may include one or more recesses 114 in one or more layers of the laminated non-conductive and conductive layers 108 and 110, and one or more electronic components 112 may be embedded in the one or more recesses 114. In some non-limiting embodiments, the one or more recesses 114 may be formed by creating one or more blind holes in the surface of PCB 106 (e.g., using sequential lamination techniques and/or precision backdrilling (laser or mechanical)). The one or more blind holes may reach down to a conductive layer 110. The electronic components 112 may be completely or partially recessed in the PCB 106.

In some embodiments, one or more electronic components 112 embedded in one or more recesses 114 may be coated with a conformal coating 116, which may be, for example and without limitation, parylene, acrylic, epoxy, urethane, silicone, polydimethylsiloxane (PDMS), SU-8, or benzocyclobutene (BCB). In some embodiments, the conformal coating 116 may be one of the non-conductive layers 108 of the PCB 106. In some embodiments, the conformal coating 116 may be configured to fluidically isolate the electronic component 112 from fluid in the microfluidic feature 104. In some embodiments, the microfluidic layer 102 may be attached to the conformal coating 116 (see FIG. 1). In some embodiments, the conformal coating 116 may planarize a surface of the PCB 106 to which the microfluidic layer 102 is attached. In some embodiments, the conformal coating 116 may fill all or a portion of the one or more recesses 114 not filled by electronic components 112.

One or more chemical reactions can be performed in microfluidic features 104, such as, for example, one or more channels and/or wells of the microfluidic layer 102. In some embodiments, the reactions may include a nucleic acid amplification reaction, of which polymerase chain reaction (PCR) is one example. Additional amplification reactions are well known to those of skill in the art. Thermal melting analysis of amplified nucleic acids can be performed after completion of nucleic acid amplification in the microfluidic features 104 formed in the microfluidic layer 102. The electronic component 112 may be configured to control the reactions performed in the microfluidic layer 102. Specifically, in one embodiment, to perform an amplification reaction, for instance PCR, in the microfluidic layer 102, the electronic component 112 may be configured to cycle the temperature in one or more microfluidic features 104 according to a PCR thermal profile. In yet another embodiment, the electronic component 112 may be configured to ramp, or increase at a consistent rate, the temperature in one or more microfluidic features 104 to generate a nucleic acid thermal melting curve. In some embodiments, an optical system may be included in the electronic component 112 to monitor an amplification reaction and/or thermal melting reaction and generate a melting curve for nucleic acids in one or more microfluidic features 104. A flow control circuitry may additionally be provided as part of the electronic component 112 to control the fluid flow between the microfluidic features of the microfluidic layer 102.

In some embodiments, the one or more microfluidic features 104 may have one or more micro-scale (e.g., approximately 100 um or less) dimensions, which may enable rapid heating of fluid in the microfluidic features 104 and/or small reaction volumes. In some embodiments, one or more electronic components 112 may be large relative to the one or more microfluidic features 104. In some embodiments, the one or more recesses 114 may allow one or more relatively large electronic components 112 to be embedded in the one or more recesses 114 without affecting the one or more micro-scale dimensions of the one or more microfluidic features 104.

In some embodiments, the one or more electronic components 112 may be off-the-shelf (OTS) components, which may be inexpensive (e.g., less than 1 cent per component). The OTS components may be small (e.g., having sizes from 100's of μm to several mm) but may still be large relative to the one or more microfluidic features 104, which may, for example and without limitation, have one or more dimensions between 10 μm and 100 μm. In some embodiments, the one or more recesses 114 may enable OTS components, which would otherwise be incompatible with microfluidic devices due to their large size, to be compatible with the microfluidic device 100.

Although in some embodiments, as described above, one or more electronic components 112 may be embedded in one or more recesses 114, this is not required. In some alternative embodiments, one or more electronic components 112 may be formed or placed in the PCB 106. For instance, in some embodiments, one or more passive components (e.g., resistors or capacitors) may be formed in the PCB 106 by, for example and without limitation, adding one or more materials (e.g., resistive or capacitive materials) to the structure of PCB 106 to create the electronic component 112. In some embodiments, one or more electronic components 112 may be placed in the PCB 106 by, for example and without limitation, placing one or more active or passive components (e.g., resistors, capacitors, diodes, transistors, or integrated circuits) on an internal layer (e.g., a conductive layer 110) of the PCB 110 and then burying the one or more placed components as additional layers are added to the PCB 106.

FIG. 2 is a cross-sectional view of an example of a microfluidic device 100 where the one or more electronic components 112 include one or more formed or placed components 218 according to some embodiments. In the embodiment illustrated in FIG. 2, the components 218 are resistors formed in the PCB 206. In some embodiments, the formed resistors may be used to heat and/or sense the temperature of fluid in the one or more microfluidic features 104. In some embodiments, the resistors heat and/or sense the temperature during amplification and thermal melting analysis.

In some embodiments, as illustrated in FIG. 2, the PCB 106 may include one or more conductive layers 110 above the one or more formed or placed components 218. However, this is not required, and, in some alternative embodiments, the top conductive layer 110 may be etched away. In some embodiments, as illustrated in FIG. 2, the PCB 106 may include a separate adhesion layer 220, which attaches the microfluidic layer 102 to the PCB 106. However, this is not required, and, in some alternative embodiments, the microfluidic layer 102 may be attached to a non-conductive layer 108 (e.g., a pre-preg layer).

In some non-limiting embodiments, the PCB 106 may include one or more electronic components 112 embedded in one or more recesses 114 and one or more electronic components 112 formed or placed in the PCB 106.

In some embodiments, one or more of the conductive layers 110 may be made with copper (e.g., copper having a 0.5, 1, or 2 oz copper thickness). In some non-limiting embodiments, one or more of the conductive layers 110 may be made with heavy copper (i.e., copper having a 3 oz copper thickness or greater). In some non-limiting embodiments, one or more of the conductive layers 110 may be made with extreme copper (i.e., copper having a 20-200 oz copper thickness). In some embodiments, the heavy or extreme copper may enhance the conductivity of the PCB plane, and the PCB 106 of the microfluidic device 100 may act as an integrated heat spreader. In some embodiments, the heavy or extreme copper may spread heat to one or more microfluidic features 104 of the microfluidic layer 102 attached to the PCB. In some embodiments, the heavy or extreme copper may eliminate issues associated with bonding a non-integrated heat sink/spreader to the microfluidic device 100, such as, for example, void hotspots and/or delamination. In some non-limiting embodiments, the heavy or extreme copper may spread heat provided by an internal heating component (e.g., a recessed, formed, or placed electronic component embedded in the PCB 106) or by an external heating component (e.g., a lamp, a laser, a hot plate, or a Peltier device)).

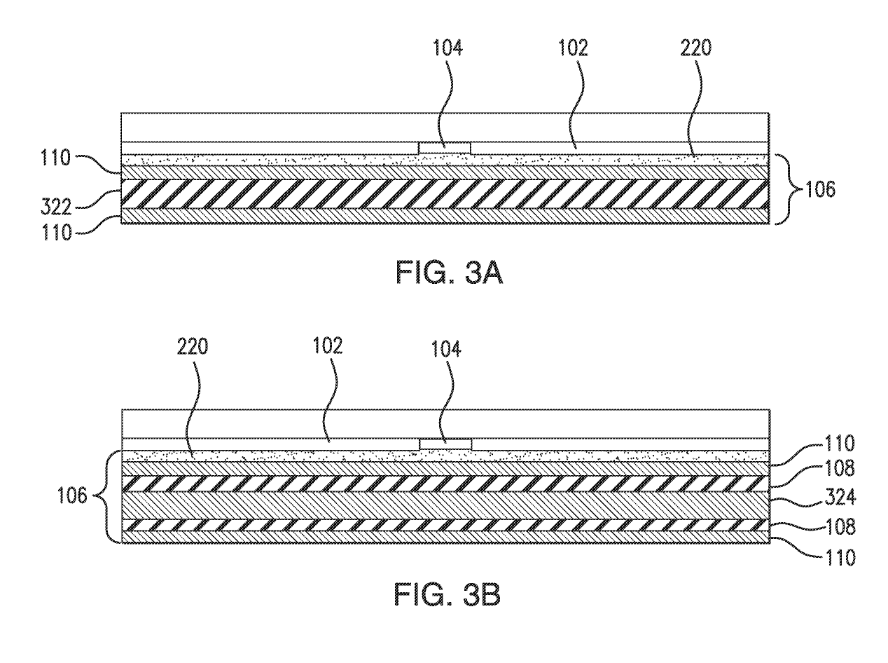

In some non-limiting embodiments, as illustrated in FIG. 3A, the PCB 106 may include an epoxy or fiberglass core 322. However, this is not required, and, in some alternative embodiments, the PCB 106 may include a metal core 324, as illustrated in FIG. 3B. In some non-limiting embodiments, the metal core 324 may be, for example and without limitation, an aluminum or copper metal core. An aluminum core may be preferred in embodiments where the microfluidic device 100 is disposable. In some embodiments, the metal core 324 may act as an integrated heat spreader. In some embodiments, the metal core 324 may spread heat to one or more microfluidic features 104 of the microfluidic layer 102 attached to the PCB. In some embodiments, the metal core 324 may eliminate issues associated with bonding a non-integrated heat sink/spreader to the microfluidic device 100. In some non-limiting embodiments, the metal core 324 may spread heat provided by an internal heating component or by an external heating component.

In some non-limiting embodiments, the metal core 324 or heavy or extreme copper could be used to spatially separate heating and temperature measurement from one or more microfluidic features 104. For example, in one-non-limiting embodiment, a single heating component may be used to heat multiple microfluidic features 104, with the metal core/heavy copper effectively spreading the heat to multiple microfluidic features 104. Similarly, in another non-limiting embodiment, the temperature sensing component (e.g., RTD) may additionally or alternatively be remote from the microfluidic feature 104. This may give the microfluidic device designer more freedom in, for example, placing the channels, reaction wells, and thermal components.

In some embodiments, the microfluidic layer 102 may be attached to the PCB 106 such that one or more microfluidic features 104 are associated with one or more electronic components 112. In some embodiments, the microfluidic layer 102 may be attached to the PCB 106 such that one or more electronic components 112 are in vertical alignment with one or more microfluidic features 104. In some embodiments, the microfluidic layer 102 may be attached to the PCB 106 such that one or more electronic components 112 are beneath one or more microfluidic features 104. In some embodiments, the microfluidic layer 102 may be attached to the PCB 106 such that one or more electronic components 112 are in close proximity to one or more microfluidic features 104. In some embodiments, one or more electronic components 112 may be separated from one or more microfluidic features 104 by only a non-conductive layer 108 (e.g., a conformal coating 116 or a pre-preg layer).

In some embodiments, one or more electronic components 112 may have a functional relationship with one or more microfluidic features 104. In some embodiments, one or more electronic components 112 may be configured to heat fluid in one or more microfluidic features 104. For example, in some non-limiting embodiments, the one or more electronic components 112 may include one or more OTS chip resistors in a recess 114 and coated by a conformal coating 116, which may act as a passivation layer, and the one or more OTS chip resistors may be configured to rapidly heat one or more microfluidic features 104. For another example, in some non-limiting embodiments, the one or more electronic components 112 may include one or more formed or placed resistors buried in the stack of laminated non-conductive and conductive layers 108 and 110, and the one or more formed or placed resistors may be configured to rapidly heat one or more microfluidic features 104. In some additional examples, one or more electronic components 112 may be configured to rapidly cycle the temperature of one or more microfluidic features 104 according to a PCR (or other amplification) profile to amplify nucleic acids in one or more microfluidic features 104. The electronic components 112 may be configured to subsequently ramp the temperature in the one or more microfluidic features 104 to generate a thermal melting curve for the amplified nucleic acids.

In some embodiments, one or more electronic components 112 may be configured to detect the temperature of fluid in one or more microfluidic features 104. For example, in some non-limiting embodiments, the one or more electronic components 112 may include one or more temperature measurement devices (e.g., thermistors or RTDs), and the one or more temperature measurement devices may be configured to detect the temperature of fluid in one or more microfluidic features 104. In some embodiments, one or more electronic components 112 may be configured to heat fluid in one or more microfluidic features 104 and to detect the temperature of the fluid in the one or more microfluidic features 104. In other embodiments, the temperature of the fluid in the one or more microfluidic features 104 may be detected to control amplification and thermal melting analysis.

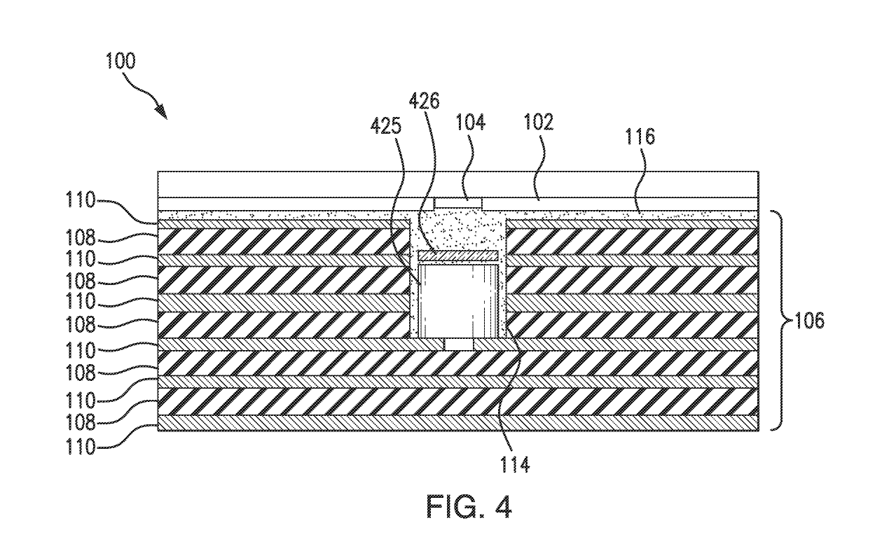

In some embodiments, the one or more electronic components 112 may be configured to emit light to or detect light from one or more microfluidic features 104. In some non-limiting embodiments, a microfluidic device 100 may include an optical system embedded in the PCB 106. For instance, in some non-limiting embodiments, the one or more electronic components 112 may include one or more optical components 425, such as, for example and without limitation, a light source (e.g., an LED) and/or a photodetector (e.g., a photodiode, phototransistor, photoresistor or other photosensitive element)(see FIG. 4). In some non-limiting embodiments, one or more optical components 425 may be embedded in one or more recesses 114 in one or more layers of the laminated non-conductive and conductive layers 108 and 110. In some non-limiting embodiments, one or more optical filters 426 may be embedded with the one or more optical components 425 in the one or more recesses 114, as illustrated in FIG. 4. In some embodiments, the PCB 106 may include a conformal coating 116, which may fluidically isolate the optical components 425 from the one or more microfluidic features 104 and may provide a planar surface to which the microfluidic layer 104 may be attached. In some non-limiting embodiments, space in the one or more recesses 114 not filled by the one or more optical components 425 and/or one or more optical filters 426 may be filled by void space or by the conformal coating 116.

In some embodiments, the one or more optical components 425 may include one or more light sources configured to emit light to one or more microfluidic features 104. In some non-limiting embodiments, the light source may be configured to excite a fluorophore in the one or more microfluidic features. In some embodiments, the one or more optical components 425 may additionally or alternatively include one or more photodetectors configured to detect light received from one or more microfluidic features 104. In some embodiments, the optical system including the one or more optical components 425 and/or one or more appropriate optical filters 426 may be configured to perform fluorescence imaging and may use very low power to do so. In some embodiments, the one or more optical components 425 of optical system embedded in the PCB 106 may be low cost and/or low power optical components 425, and the optical system embedded in the PCB 106 may have built-in alignment of the one or more optical components 425 and/or one or more appropriate optical filters 426 to the one or more microfluidic features 104. In some additional embodiments, the optical components 425 may be configured to acquire images of one or more microfluidic features 104, including channels and/or wells, during amplification and thermal melting analysis. In further embodiments, the optical components 425 may include one or more excitation sources and one or more detectors. The excitation sources may generate light at desired wavelengths to excite fluorescent labels used for detecting the amplification products during real-time PCR and thermal melting analysis by one or more detectors.

Embodiments of the present invention have been fully described above with reference to the drawing figures. Although the invention has been described based upon these preferred embodiments, it would be apparent to those of skill in the art that certain modifications, variations, and alternative constructions could be made to the described embodiments within the spirit and scope of the invention.

What is claimed is:

1. A microfluidic device comprising:
a microfluidic layer including a microfluidic feature; and
a printed circuit board (PCB) to which the microfluidic layer is attached, the PCB comprising:
electrically non-conductive layers;
electrically conductive layers laminated with the non-conductive layers; and
an electronic component embedded in the laminated non-conductive and conductive layers, wherein a non-conductive layer of the non-conductive layers is configured to fluidically isolate the electronic component from fluid in the microfluidic feature, and the electronic component is connected to a conductor of a conductive layer of the conductive layers.

2. The microfluidic device of claim 1, wherein the PCB further includes a recess in one or more layers of the laminated non-conductive and conductive layers, and the electronic component is embedded in the recess.

3. The microfluidic device of claim 2, wherein the non-conductive layer configured to fluidically isolate the electronic component from fluid in the microfluidic feature is a conformal coating.

4. The microfluidic device of claim 3, wherein the microfluidic layer is attached to the conformal coating.

5. The microfluidic device of claim 3, wherein the conformal coating is configured to planarize a surface of the PCB to which the microfluidic layer is attached.

6. The microfluidic device of claim 3, wherein the electronic component is large relative to the microfluidic feature.

7. The microfluidic device of claim 2, wherein recess includes one or more optical filters.

8. The microfluidic device of claim 1, wherein the electronic component is a formed passive component, a placed discrete passive component, or a placed active component.

9. The microfluidic device of claim 8, wherein the electronic component is a resistor, capacitor, diode, transistor, or integrated circuit.

10. The microfluidic device of claim 1, wherein the electronic component is configured to heat fluid in the microfluidic feature.

11. The microfluidic device of claim 1, wherein the electronic component is a light source configured to emit light and irradiate the microfluidic feature.

12. The microfluidic device of claim 11, wherein the light source is configured to excite a fluorophore in the microfluidic feature.

13. The microfluidic device of claim 1, wherein the electronic component is a photodetector configured to detect light received from the microfluidic feature.

14. The microfluidic device of claim 1, wherein the electronic component is configured to measure the temperature of fluid in the microfluidic feature.

15. The microfluidic device of claim 1, wherein the microfluidic feature includes a microfluidic channel.

16. The microfluidic device of claim 1, wherein the microfluidic feature includes a microwell.

17. The microfluidic device of claim 1, wherein the electronic component is below the microfluidic feature.

18. The microfluidic device of claim 1, further comprising an adhesion layer.

19. The microfluidic device of claim 1, further comprising a plurality of microfluidic layers.

20. The microfluidic device of claim 1, wherein the microfluidic layer includes a plurality of microfluidic features.

21. The microfluidic device of claim 1, wherein the PCB includes a plurality of electronic devices.

22. The microfluidic device of claim 21, wherein the plurality of electronic devices includes a light source and a photodetector.

23. The microfluidic device of claim 22, wherein the light source and photodetector are embedded in one or more recesses in one or more layers of the laminated non-conductive and conductive layers.

24. The microfluidic device of claim 23, wherein the recess includes one or more optical filters.

25. The microfluidic device of claim 1, wherein one or more of the conductive layers comprises copper have greater than or equal to a 3 oz thickness.

26. The microfluidic device of claim 1, wherein the microfluidic layer is attached to the PCB using solvent, adhesive, or thermal bonding.

27. The microfluidic device of claim 1, wherein the PCB is a metal core PCB.

28. A microfluidic device comprising:
a microfluidic layer including one or more microfluidic features;
a metal core printed circuit board (PCB) to which the microfluidic layer is attached, the PCB comprising:
electrically non-conductive layers;
electrically conductive layers laminated with the non-conductive layers; and
a metal core configured to spread heat to the one or more microfluidic features.

29. The microfluidic device of claim 28, further comprising a component connected to the metal core and configured to provide the heat spread by the metal core.

30. The microfluidic device of claim 29, wherein the component is embedded in the laminated non-conductive and conductive layers of the PCB.

31. The microfluidic device of claim 30, wherein the PCB further includes a recess in one or more layers of the laminated non-conductive and conductive layers, and the component is embedded in the recess.

32. The microfluidic device of claim 28, wherein the heat spread by the metal core is provided by a component external to the microfluidic device.

33. The microfluidic device of claim 28, wherein the microfluidic feature includes a microfluidic channel.

34. The microfluidic device of claim 28, wherein the microfluidic feature includes a microwell.

35. A method of manufacturing a microfluidic device, the method comprising:
embedding an electronic component in laminated electrically non-conductive layers and electrically conductive layers of a printed circuit board (PCB), wherein the electronic component is connected to a conductor of a conductive layer of the conductive layers; and
attaching a microfluidic layer including a microfluidic feature to the PCB, wherein the electronic component is fluidically isolated from fluid in the microfluidic feature by a non-conductive layer of the non-conductive layers.

36. The method of claim 35, wherein embedding the electronic component comprises:
forming a recess in one or more layers of the laminated non-conductive and conductive layers; and
embedding the electronic component in the recess.

37. The method of claim 36, wherein embedding the electronic component comprises forming a conformal coating on the PCB, wherein the non-conductive layer configured to fluidically isolate the electronic component from fluid in the microfluidic feature is the conformal coating.

38. The method of claim 37, wherein attaching the microfluidic layer to the PCB comprises attaching the microfluidic layer to the conformal coating.

39. The method of claim 35, wherein embedding the electronic component comprises forming or placing the electronic component in the PCB.

40. The method of claim 35, wherein one or more of the conductive layers comprises copper have greater than or equal to a 3 oz thickness.

41. The method of claim 35, wherein the PCB is a metal core PCB.

42. A method of heating fluid in a microfluidic feature of a microfluidic device comprising a microfluidic layer including the microfluidic feature and a printed circuit board (PCB) to which the microfluidic layer is attached, the method comprising:
using a first electronic component embedded in laminated electrically non-conductive layers and electrically conductive layers of the PCB to heat fluid in the microfluidic feature of the microfluidic device, wherein the first electronic component is fluidically isolated from the fluid in the microfluidic feature by a non-conductive layer of the non-conductive layers, and the first electronic component is connected to a conductor of a conductive layer of the conductive layers.

43. The method of claim 42, further comprising using the first electronic component to measure the temperature of the fluid in the microfluidic feature.

44. The method of claim 42, further comprising using a second electronic component to measure the temperature of the fluid in the microfluidic feature.

45. A method of irradiating fluid in a microfluidic feature of a microfluidic device comprising a microfluidic layer including the microfluidic feature and a printed circuit board (PCB) to which the microfluidic layer is attached, the method comprising:
using a light source embedded in laminated electrically non-conductive layers and electrically conductive layers of the PCB to emit light and irradiate the fluid in the microfluidic feature of the microfluidic device, wherein the light source is fluidically isolated from the fluid in the microfluidic feature by a non-conductive layer of the non-conductive layers, and the light source is connected to a conductor of a conductive layer of the conductive layers.

46. The method of claim 45, wherein irradiating the fluid comprises exciting a fluorophore in the microfluidic feature.

47. The method of claim 45, further comprising using a photodetector embedded in the laminated non-conductive and conductive layers of the PCB to detect light received from the microfluidic feature.

48. A method of manufacturing a microfluidic device, the method comprising:
attaching a microfluidic layer including a microfluidic feature to a metal core printed circuit board (PCB) including electrically non-conductive layers, electrically conductive layers laminated with the non-conductive layers, and a metal core configured to spread heat to the one or more microfluidic features.

49. A method of spreading heat to fluid in one or more microfluidic features of a microfluidic device comprising a microfluidic layer including the one or more microfluidic feature and a printed circuit board (PCB) to which the microfluidic layer is attached, the method comprising:
using a metal core of the printed circuit board (PCB) to spread heat to the one or more microfluidic features, wherein the PCB includes the metal core, electrically non-conductive layers, and electrically conductive layers laminated with the non-conductive layers.

50. The microfluidic device of claim 1, wherein the electronic component controls a reaction within the microfluidic feature.

51. The microfluidic device of claim 50, wherein the reaction is selected from the group comprising: nucleic acid amplification, thermal melting analysis, or a combination thereof.

52. The microfluidic device of claim 28, wherein the electronic component controls a reaction within the microfluidic feature

53. The microfluidic device of claim 52, wherein the reaction is selected from the group comprising: nucleic acid amplification, thermal melting analysis, or a combination thereof.
